SEQUENCE LISTING (6 sequences)

SEQ ID NO: 1 - DNA, 1368 bp, Physaria fendleri
  source 1..1368: mol_type = genomic DNA
atgattcaatgggtgatggtttcagttcttcagttagcagagctaacagtgagctccattgttcatattctatatggactttacatatttacctccgccgtagccggagatctcactcagtcttgggttgaatcaatcttcaaacccaaaaccaccattgaagtcaaagaaacagtccgagaaaagatgacagatgttaatgatttacctcccattgttttagtccatgggatttttggatttggcaaaggaagattaggtggtttatcatactttggtggagcagagaagaaggatgagagagttttagttcctgatttaggatctttgacgagtgtacacgatagggctagagaattgttttattatctgaaaggtggagtagttgactatggagaagaacatagtaaagcttgtggacattctcgatttggtcgtttatatgaaaaaggggaataccaagaatgggatgaagatcacccaattcactttgttggtcactctgctggtgctcaagttgttcgtctcattcagcaaatgctcgctgacaagatgtttgagggttatgaggacacaaatgagaactgggtattgagtttaacatcattgtcaggagcattaaacgggtgtactagaacctactttgatggacttcagcctgaagatgggaagtccatgaaatctatatctctgcttcagatttgcagacttggaaccatattgtatgactggattgacagttcttggctcaaatcttattacaatttcgggttcgaccatttcaacatgtcgagaaagaagatcggttttcgcggactagtagattgtcttcttggaaatgctggtccttatgcatctggagattggatcttgcctgatctctcaatccagggatcgatgaagctcaatgctggtctcaagacttttccaaacacattctacttcagctatgcaactaagcgtagtagaaaacctctcgggatgggaatacatcctctgcttttcatccgcgtgttgcaaatgagtcattggcgatttcctcgggatcttcctctgccttataaaggatacagagatgaagattggcatgacaatgatggagcagtgaacactatatcaatgactcacccgcgaattcccatcgaacattcgagtctcagtcttcatagtgactcggactctatcccgcttcaaccaggcatttggtactacaagatcgtggaagcagatcatatcatgttcatactaaaccgagacagagcaggcgtggagttcgatctgatatacgacagtatcttcgagcgttgcaggaaacatgtaaccagaaagatcacccagacattaccaaacgaagctaaacaccagtaa

SEQ ID NO: 2 - protein, 455 aa, Physaria fendleri
MIQWVMVSVLQLAELTVSSIVHILYGLYIFTSAVAGDLTQSWVESIFKPKTTIEVKETVREKMTDVNDLPPIVLVHGIFGFGKGRLGGLSYFGGAEKKDERVLVPDLGSLTSVHDRARELFYYLKGGVVDYGEEHSKACGHSRFGRLYEKGEYQEWDEDHPIHFVGHSAGAQVVRLIQQMLADKMFEGYEDTNENWVLSLTSLSGALNGCTRTYFDGLQPEDGKSMKSISLLQICRLGTILYDWIDSSWLKSYYNFGFDHFNMSRKKIGFRGLVDCLLGNAGPYASGDWILPDLSIQGSMKLNAGLKTFPNTFYFSYATKRSRKPLGMGIHPLLFIRVLQMSHWRFPRDLPLPYKGYRDEDWHDNDGAVNTISMTHPRIPIEHSSLSLHSDSDSIPLQPGIWYYKIVEADHIMFILNRDRAGVEFDLIYDSIFERCRKHVTRKITQTLPNEAKHQ

SEQ ID NO: 3 - DNA, 1512 bp, Physaria fendleri
  source 1..1512: mol_type = genomic DNA
atgtcgattttggattctggaggtgttagtacgacggatacggagtacgatggcggagataagcttcgtagacggaaatctaaatcggattccaccggacttctttctgactccactgtatcagatgatgttagggatcggattgattccgttgctaacgaagacgctcaggggttgagcggagataacggtagtgataccgagattagggaaattcgtggagcaagaagcggtgggggaggaagaggaaacagtaatgctacgtacgctcgaccttcggttccagctcatcggaaggcgaaagagagtcctctcagctccgacgcaatcttcaagcagagtcatgcaggattattcaacttgtgtgttgttgttcttgttgctttaaacagtagactcatcatcgaaaatctgatgaagtatggttggttgatccgaacggatttctggtttagttcaacatcactgcgagattggccgcttttcatgtgttgtctctccctttcgatctttcctttggcttcctttaccgttgagaaattggtacttcagaagctcatatctgagcctgttgtcatcattcttcatattattatcaccacgacagcggttttgtatccagtttacgtcaccctaaggtgtgattctgccttcttatcaggtgtcacattgatgctcctcacttgcattgtgtggctaaagttggtttcttatgctcacacgaactacgacataagaaccctagccaattcagctgataaggccaatcctgaagtctcctacgacgttagcttcaagagcttggcatatttcatggttgctcccacactgtgttatcagccaagctatccacgttctccgtgtatccggaagggttgggtggctcgtcaatttgcaaagcagatcatattcactggattcatgggatttataatagaacaatatacaaaccctattgttaggaactcaaagcatccttttaaaggggatcttctatatgctattgaaagagtgttgaagctttcaataccaaatttatatgtgtggctctgcatgttctactgcttcttccacctttggttaaacgtattggcagagcttctctgcttcgcggatcgtgagttctacaaagattggtggaatgcaaaaagtgtgggagattattggagaatgtggaatatgcctgttcataaatggatggttagacatatatacttcccgtgcctgcgcaacaagatacccaagtcaatcgccgttatcattgctttcttagtctctgcagtctttcatgagctatgcatcgcggtcccttgccgcctcttcaggttatgggcttttatggggattatgtttcaggtgcctttggtctttatcacaaactatctacaagaaaggttcggctcaacggtgggcaacatgatcttctggttcatcttctgcattttcggacaacctatgtgtgtgcttctttattaccacgacctgatgaaccgcaaaggatcaacctcatga

SEQ ID NO: 4 - DNA, 948 bp, Physaria fendleri
  source 1..948: mol_type = genomic DNA
atgggcgaatccagagaattccaatctaagaaagcttcaaatcaattccactccatcatcaccatgattatctggctcggcaccattcacttaaacgtcgctattgttctcttttctctcattttccttcctccttctctatctctcctggtcttcggcttcctctctgtatttatcttcctcccaatcgatgatcgtagcaaatacggtcgtaagcttgctaggtacttatgtaagcattcatgtacttatttccctgtctctcttcacgtcgaggattacgatgcttttcaatctgatcgtgcctacgtgtttggttatgaaccacattcggtgttaccgatcggtgttgttgccctttgtgatctcacagggtttatgcctctgaataagatcaaagttcttgctagttctgctattttctacactccctttctaaggcacatatggtcatggttagggctcacccctgcttctaggaggaatttcacttcccttttggattctggctatagttgtgttcttatacctggtggtgtccaggagacttatcatatgaaacatgacgttgagaccgtctttctttcctcgagaagaggatttgtgcgcatagccatggaacatgggtgtcctatagttccagttttctgctttgggcagtcacgggtgtacagttggtggaagccagattgggatctctatcttaaattatcaagagcaatcaggtttaccccaatctgcttctggggagtttttggaaccccattaccgtatcgagagcctatgcatgtggtagttggtaaaccaatagaagttacaaaaactctgcagccgagcgatgaagaggtagctaagtatcatgggcagtttgtggaagcacttagggatctggttgagaggcacaaggccagagtaggctattccgatcttcagttgaaaatactttga

SEQ ID NO: 5 - DNA, 1566 bp, Ricinus communis
  source 1..1566: mol_type = genomic DNA
atgacgattctcgaaacgccagaaactcttggcgtcatctcctcctccgccacttccgatctcaacctctctctccgacgtagacggacctcaaatgactccgatggtgcacttgctgatttggcttcgaagtttgatgatgatgacgacgtaagatcggaagattctgctgaaaatattatcgaagatcctgtagcagcggttactgaattggcgacagcaaagagtaacggaaaagactgtgttgccaatagtaataaggataaaattgatagccatggaggatcatcggattttaaacttgcatataggccttcggttccagctcaccggtcacttaaggagagtccgcttagctctgatttaatatttaaacaaagtcatgcaggtctgtttaacctttgtatagtagtgctcgtagctgttaacagcaggctcatcattgagaatttaatgaagtatggctggttaattaagacgggcttttggtttagttcaagatcattgagagattggccgctttttatgtgctgtctttctctcccagtattcccccttgctgcctatctagttgagaaggccgcatatcgaaaatatatatctccgcctattgttattttccttcatgtgatcatcacctcagcagctgttttgtacccagcttctgtaattctcagttgtgaatctgcttttttatctggtgtcacattgatggaacttgcttgtatggtatggttgaaattggtatcctatgcacatacaaactatgatatgagagcgatcgctgacaccattcataaggaagatgcatccaattcttctagtacagagtattgtcatgatgtgagctttaagactttggcgtacttcatggtcgcacccacattatgttaccagccaagttatcctcgcacagcatttattagaaagggctgggtgttccgtcaatttgtcaaactaataatttttacaggattcatgggatttatcatagaacaatacatcaatcctatcgtccagaattctcaacaccctttaaaaggggatctcttatatgccattgagagggttctgaagctctcagttccgaatttatatgtgtggctctgcttgttctactgcttttttcacctgtggttgaatatagttgctgagctccttcgcttcggtgaccgggagttctacaaagattggtggaatgcaaaaactgttgaggagtactggaggatgtggaatatgcctgttcacaagtggatggttcgccatatctacttcccatgcctacgtcgtaaaataccaaggggggtagcaatagttattgctttcttcgtttcagctgtatttcatgagttgtgcattgctgttccttgccacatgttcaaactttgggctttttttggaataatgtttcagattcctttagttgtgatcactaattattttcaaaggaagttcagaagctcaatggtgggaaatatgatcttctggttctttttctgcattctcggccaacctatgtgtgtactgttgtattaccatgacctaatgaatcgcgatgggaactga

SEQ ID NO: 6 - DNA, 1023 bp, Ricinus communis
  source 1..1023: mol_type = genomic DNA
atgggggaagaagcgaatcataataataataataataatatcaatagtaatgatgagaagaatgaagagaaatcaaattatacagttgtaaattcgagagaactatacccaacgaacatatttcacgcactgttagcgttgagcatatggattggttcaatccatttcaatctcttcttactcttcatctcttatctcttcctttcttttcccacattcctcctgattgttggattttttgtggtgttaatgttcattccgatcgacgaacacagtaagttgggccgtcgtttgtgcaggtatgtatgcagacatgcgtgcagtcattttccggtaactctccatgttgaagacatgaatgcttttcattctgatcgtgcttacgtttttggttatgagccacattcagtatttccccttggtgtttctgtactatcagatcactttgctgtcctgccccttcctaaaatgaaggtccttgcaagtaacgctgtgtttcgcacaccagttttaaggcatatatggacatggtgtggtcttacatcagcaacaaagaaaaatttcactgccctcctagcatctggttatagttgcattgtgattcccggtggagttcaagagacattttatatgaagcatggctctgagattgctttccttaaggcgagaagagggtttgtccgagtagctatggagatggtgaaacccttggttccagttttctgctttggtcaatcgaacgtgtacaagtggtggaaacctgatggcgagttatttatgaaaattgctagagctattaagttcagcccaattgtcttttggggagttctcggttctcatttaccgctacaacgtccgatgcatgttgtcgtcggtaaaccgattgaggtgaagcaaaatccacagcctacagtggaagaggtctcagaagtacagggtcagtttgttgcggcacttaaagatctttttgaaaggcataaagcacgggttggctatgcagaccttacacttgaaattctttga

(19) United States
(12) Patent Application Publication
(10) Pub. No.: US 2024/0344076 A1
(43) Pub. Date: Oct. 17, 2024

(54) METHOD AND MODIFIED ORGANISMS FOR BIOSYNTHESIS OF TARGET FATTY ACIDS

(71) Applicant: Washington State University, Pullman, WA (US)

(72) Inventors: Philip D. Bates, Pullman, WA (US); Sajina Bhandari, Pullman, WA (US); Prasad Parchuri, Pullman, WA (US); Abdul Azeez, Pullman, WA (US)

(21) Appl. No.: 18/632,652

(22) Filed: Apr. 11, 2024

Related U.S. Application Data
(60) Provisional application No. 63/496,365, filed on Apr. 14, 2023.

(51) Int. Cl. C12N 15/82; C12N 9/10; C12N 9/20; C12P 7/42

(52) U.S. Cl. CPC C12N 15/8247; C12N 9/1029; C12N 9/20; C12P 7/42; C12Y 203/0102; C12Y 301/01003

(57) ABSTRACT

Method and associated transgenic oil-producing plants, protists, bacteria, and fungi for increasing concentration of target fatty acids for use in food, biofuel, or industrial chemical applications. Modification of organisms to enhance lipase expression induces triacylglycerol (TAG) remodeling to change the composition of stored oils. Lipase converts endogenously synthesized TAG back to diacylglycerols, allowing resynthesis into TAG with different fatty acid compositions using fatty acid assembly enzymes either native to the organism or expressed through additional transgenic modification.

### CROSS-REFERENCE TO RELATED APPLICATIONS

This application claims the benefit of U.S. Provisional Patent Application 63/496,365 filed on Apr. 14, 2023, and incorporates said provisional application by reference into this document as if fully set out at this point.

### FEDERALLY SPONSORED RESEARCH

This invention was made with government support under grant numbers 2020-67013-30899 and 2016-67013-29020 awarded by the United States Department of Agriculture through the National Institute of Food and Agriculture. The government has certain rights in the invention.

### SEQUENCE LISTING

This application includes as the Sequence Listing the complete contents of the accompanying XML format file ‘2024-04-11_12770140AA_seqlisting.xml’, created Mar. 5, 2024, containing 3,595,356 bytes, hereby incorporated by reference.

### TECHNICAL FIELD

This disclosure relates to transgenic bioengineering to increase production of target fatty acids for food, biofuel, or industrial chemicals, within oilseeds and other oil-producing plants, protists, bacteria, and fungi.

### BACKGROUND

Specific oil composition can be more important than total oil quantity, in selecting, growing, and modifying feedstock for particular purposes. For nutritional use, not all fatty acids have equal health effects, so food producers may favor concentration of beneficial fats such as certain Omega-3s over others which may be more associated with adverse health consequences. Similarly, for non-food industries, different fatty acids are suited for different applications. Hydroxy fatty acids (HFA) contained in triacylglycerols (TAG) are an important chemical precursor for various industrial and consumer products such as lubricants, paints, resins, biodegradable polymers, biofuels, pharmaceuticals, perfumes, and cosmetics.

Castor beans (Ricinus communis) are a significant farmable source of HFA, but cultivating R. communis is prohibited in some jurisdictions because of potential use in producing dangerous concentrations of ricin poison. Lesquerella (Physaria fendleri) is a non-toxic and native North American source of seed oil which contains HFA as a portion of its TAG, though P. fendleri has not yet been bred as a high yielding crop. Lipid biosynthetic genes from R. communis and P. fendleri have been studied using bioengineering to better understand the function of genes involved in lipid biosynthesis and other biochemical processes.

Researchers have made transgenic Arabidopsis and Camelina lines expressing different oil biosynthesis genes from those in native plants, but not yet with comparable amounts of HFA as R. communis or P. fendleri. Recently, it has been recognized that oil accumulation within oil-synthesizing organisms is dynamic and not a metabolic endpoint, a phenomenon coined TAG remodeling. Bhandari S, Bates PD (2021) Triacylglycerol remodeling in Physaria fendleri indicates oil accumulation is dynamic and not a metabolic endpoint. Plant Physiol 187:799-815. This process changes the composition of the oil (i.e., proportions of constituent fatty acids) after it is synthesized and provides a potential way to accumulate more unusual and valuable fatty acids. TAG remodeling can be contrasted with linear pathways such as the Kennedy pathway, and two major pathways for production of diacylglycerol (DAG), the immediate precursor to TAG, which have been identified in plants: de novo DAG synthesis and conversion of the membrane lipid phosphatidylcholine (PC) to DAG, with each pathway producing distinct TAG compositions.

Despite earlier recognition of TAG remodeling, researchers were not able to fully model and confirm TAG assembly pathways in unusual fatty acid accumulating plants, such as those which produce or could be modified to produce HFA. Genes involved in TAG remodeling have not previously been characterized.

### SUMMARY

Presented here are a transgenic method and associated transgenic organisms for TAG remodeling of the oil composition of oil-producing plants, protists, bacteria, or fungi to increase production of target fatty acids.

In an example embodiment, cells of a selected oil-producing species are modified with gene sequences associated with production of lipase to induce TAG remodeling. Modified specimens are selected which show overexpression of the lipase-producing genes-which can include TAG lipase like-1 (TAGL1)—and increased concentration of a target fatty acid, which can include one or more HFA, or other unusual fatty acids. Alternatively, cells can be modified with additional gene sequences associated with production of at least one triacylglycerol biosynthetic assembly enzyme, which can include DAG acyltransferase 1 (DGAT1) and DAG acyltransferase 2 (DGAT2). The modified oil-producing organism may further exhibit one or more of increased omega-3 fatty acids, decreased omega-6 fatty acids, and reduced saturated fatty acids as compared to an unmodified control organism.

Another embodiment includes transgenic organisms produced using this method, overexpressing genes associated with lipase-production—such as TAGL1—to stimulate TAG remodeling to alter oil composition by increasing composition of target fatty acids, such as one or more HFA. Organisms can be further modified with gene sequences associated with production of at least one fatty acid modeling enzyme, which can include DGAT1 and DGAT2.

Accordingly, the present invention harnesses new understanding of TAG remodeling and provides an opportunity to expand and diversify sustainable feedstock for biosynthesis of HFA and other valuable fatty acids.

### BRIEF DESCRIPTION OF THE FIGURES

FIG. 1(A) depicts TAG remodeling pathway with exogenously expressed lipase and endogenous TAG reassembly.

FIG. 1(B) depicts TAG remodeling pathway with exogenously expressed TAG reassembly.

FIG. 2(A) shows total seed oil increase from engineering TAG remodeling by PfeTAGL1 expression in Arabidopsis previously engineered to produce HFA.

FIG. 2(B) shows HFA increase from engineering TAG remodeling by PfeTAGL1 expression in Arabidopsis previously engineered to produce HFA.

FIG. 3(A) shows reduction in total oil from SDP1 expression alone in Arabidopsis.

FIG. 3(B) shows increase in total oil and HFA content from PfeSDP1+RcDGAT2 expression in Arabidopsis.

FIG. 4(A) (PRIOR ART) maps shuttle vector pB34 used to clone PfeDGATs.

FIG. 4(B) (PRIOR ART) maps shuttle vector pB35 used to clone PfeTAGL1.

FIG. 4(C) (PRIOR ART) maps binary vector pB110 used to clone other gene combinations to create transgenic lines.

FIG. 5(A) maps binary vector pB110 with PfeTAGL1 and PfeDGAT1-1 expressed in head-to-head orientation.

FIG. 5(B) maps binary vector pB110 with PfeTAGL1 and PfeDGAT2 expressed in head-to-head orientation.

FIG. 5(C) maps binary vector pB110 with PfeTAGL1 and both PfeDGAT1 and PfeDGAT2 expressed in head-to-head and head-tail orientation.

FIG. 5(D) maps binary vector pB9 with PfeTAGL1 and both RcDGAT1 and RcDGAT2 expressed in head-to-head and head-tail orientation.

FIG. 6 shows fatty acid composition of TAG (without HFA) in T4 seeds expressing PfeDGAT1-1 (D1-1), PfeDGAT2 (D2), PfeTAGL1 (TL) alone, PfeTAGL1 with PfeDGAT1-1 (TLD1-1) and PfeTAGL1 with PfeDGAT2 (TLD2) in A. thaliana RcFAH line. Data are mean±SEM of three replicates for each line. Significant differences compared to control (RcFAH) are determined by two-way ANOVA and marked with asterisks [pvalue≤0.05 as (*); ≤0.01 as (**), ≤0.001 as (***), ≤0.0001 as (****)].

FIG. 7 shows fatty acid composition of 1HFA-TAG in T4 seeds expressing PfeDGAT1-1 (D1-1), PfeDGAT2 (D2), PfeTAGL1 (TL) alone, PfeTAGL1 with PfeDGAT1-1 (TLD1-1) and PfeTAGL1 with PfeDGAT2 (TLD2) in A. thaliana RcFAH line. Data are mean±SEM of three replicates for each line. Significant difference compared to control (RcFAH) are determined by two-way ANOVA and marked with asterisks [pvalue ≤0.05 as (*); ≤0.01 as (**), ≤0.001 as (***), ≤0.0001 as (****)].

FIG. 8 shows fatty acid composition of 2HFA-TAG in T4 seeds expressing PfeDGAT1-1 (D1-1), PfeDGAT2 (D2), PfeTAGL1 (TL) alone, PfeTAGL1 with PfeDGAT1-1 (TLD1-1) and PfeTAGL1 with PfeDGAT2 (TLD2) in A. thaliana RcFAH line. Data are mean±SEM of three replicates for each line. Significant differences compared to control (RcFAH) are determined by two-way ANOVA and marked with asterisks [pvalue ≤0.05 as (*); ≤0.01 as (**), ≤0.001 as (***), ≤0.0001 as (****)].

FIG. 9 shows regiochemical analysis of 1HFA-TAG species in T4 seeds expressing PfeDGAT1-1 (D1-1), PfeDGAT2 (D2), PfeTAGL1 (TL) alone, PfeTAGL1 with PfeDGAT1-1 (TLD1-1) and PfeTAGL1 with PfeDGAT2 (TLD2) in A. thaliana RcFAH line. Data are mean±SEM of three replicates for each line. Significant difference (p-value≤0.05) compared to control (RcFAH) are determined by T-test and marked with an asterisk (*).

FIG. 10 shows fatty acid composition of MAG from 1HFA-TAG molecular species in T4 seeds expressing PfeDGAT1-1 (D1-1), PfeDGAT2 (D2), PfeTAGL1 (TL) alone, PfeTAGL1 with PfeDGAT1-1 (TLD1-1) and PfeTAGL1 with PfeDGAT2 (TLD2) in A. thaliana RcFAH line. Data are mean±SEM of three replicates for each line. Significant differences compared to control (RcFAH) are determined by two-way ANOVA and marked with asterisks [p-value≤0.05 as (*); ≤0.01 as (**), ≤0.001 as (***), ≤0.0001 as (****)].

FIG. 11 shows regiochemical analysis of 2HFA-TAG species in T4 seeds expressing PfcDGAT1-1 (D1-1), PfeDGAT2 (D2), PfeTAGL1 (TL) alone, PfeTAGL1 with PfeDGAT1-1 (TLD1-1) and PfeTAGL1 with PfeDGAT2 (TLD2) in A. thaliana RcFAH line. Data are mean±SEM of three replicates for each line. Significant differences (p-value ≤0.05) compared to control (RcFAH) are determined by T-test and marked with asterisks (**).

FIG. 12 shows fatty acid composition of MAG from 2HFA-TAG molecular species in T4 seeds expressing PfeDGAT1-1 (D1-1), PfeDGAT2 (D2), PfeTAGL1 (TL) alone, PfeTAGL1 with PfeDGAT1-1 (TLD1-1) and PfeTAGL1 with PfeDGAT2 (TLD2) in A. thaliana RcFAH line. Data are mean±SEM of three replicates for each line. Significant differences compared to control (RcFAH) are determined by two-way ANOVA and marked with asterisks [p-value≤0.05 as (*); ≤0.01 as (**), ≤0.001 as (***), ≤0.0001 as (****)].

FIG. 13 is a phylogenetic tree of TAGL1 homologs. Some homologs from other Brassicaceae and non-Brassicaceae species are predicted to share PfeTAGL1 's effect on TAG remodeling. Phylogenetic tree was created by using P. Fendleri TAGL1 protein sequence as query using MEGA X software: MEGA X: Molecular Evolutionary Genetics Analysis across computing platforms.

FIG. 14 depicts the search for TAG remodeling mechanism, including initial lipase gene candidates.

FIG. 15 compares some diverse mechanisms to accumulate unique plant oils.

FIG. 16(A) shows enhancement of seed weight in Camelina sativa line (RcFAH_PfeKCS3) modified with PfeDGAT1 (D1), PfeDGAT2 (D2), and PfeTAGL1 individually.

FIG. 16(B) shows enhancement of seed oil production in C. sativa line (RcFAH_PfeKCS3) modified with PfeDGAT1 (D1), PfeDGAT2 (D2), and PfeTAGL1 individually.

FIG. 16(C) shows enhancement of HFA production in C. sativa line (RcFAH_PfeKCS3) modified with PfeDGAT1 (D1), PfeDGAT2 (D2), and PfeTAGL1 individually.

FIG. 17 is a phylogenetic tree of P. fendleri and other SDP1 protein homologs. Some homologs from other Brassicaceae and non-Brassicaceae species are predicted to share PfeSDP1's effect on TAG remodeling. The phylogenetic tree was constructed using MEGA11. Numbers at the tree branches indicate percent bootstrap support of 1000 iterations. Species abbreviations and accession numbers of each protein are provided below in Table 3.

FIG. 18(A) shows enhancement of seed oil per seed in C. sativa line (RcFAH_PfeKCS3) modified with PfeDGAT1 (D1), PfeDGAT2 (D2), and PfeTAGL1 individually, and all three together.

FIG. 18(B) shows enhancement of seed oil by weight production in C. sativa line (RcFAH_PfeKCS3) modified with PfeDGAT1 (D1), PfeDGAT2 (D2), and PfeTAGL1 individually, and all three together.

FIG. 18(C) shows seed fatty acid composition (mol %) in C. sativa line (RcFAH_PfeKCS3) modified with PfeDGAT1 (D1), PfeDGAT2 (D2), and PfeTAGL1 individually, and all three together.

FIG. 18(D) shows seed fatty acid composition as μg per seed in C. sativa line (RcFAH_PfeKCS3) modified with PfeDGAT1 (D1), PfeDGAT2 (D2), and PfeTAGL1 individually, and all three together.

FIG. 19(A) shows enhancement of seed weight in C. sativa line (RcFAH) modified with RcDGAT1, RcDGAT2, and PfeTAGL1 together.

FIG. 19(B) shows seed fatty acid composition (mol %) in C. sativa line (RcFAH) modified with RcDGAT1, RcDGAT2, and PfeTAGL1 together.

FIG. 20(A) shows enhancement of seed oil by weight in Arabidopsis RcFAH line modified with PfeDGAT1 (D1), PfeDGAT2 (D2), and PfeTAGL1 individually, and TAGL1 with either DGAT together, and all three together.

FIG. 20(B) shows seed fatty acid composition (mol %) in Arabidopsis RcFAH line modified with PfeDGAT1 (D1), PfeDGAT2 (D2), and PfeTAGL1 individually, and TAGL1 with either DGAT together, and all three together.

FIG. 20(C) shows seed fatty acid composition as per seed weight in Arabidopsis RcFAH line modified with PfeDGAT1 (D1), PfeDGAT2 (D2), and PfeTAGL1 individually, and TAGL1 with either DGAT together, and all three together.

FIG. 21(A) shows seed fatty acid composition in wild-type Arabidopsis modified with PfeTAGL1. Arrows indicate reduction in saturated fatty acid (16:0) and increase in omega-3 fatty acid (18:3).

FIG. 21(B) shows the total lipid content in Arabidopsis modified with PfeTAGL1.

FIG. 22(A) shows seed fatty acid composition in wild-type Camelina sativa modified with PfeTAGL1 (TL) together with either PfeDGAT1 (D1-1) or PfeDGAT2 (D2). Arrows highlight decreases in saturated fatty acid (16:0) and omega-6 fatty acid (18:2), and increases in oleic acid (18:1) and omega-3 fatty acid (18:3).

FIG. 22(B) shows total lipid content in Camelina sativa modified with PfeTAGL1 (TL) together with either PfeDGAT1 (D1-1) or PfeDGAT2 (D2).

### DETAILED DESCRIPTION

In the description herein, a word appearing in the singular encompasses its plural counterpart, and a word appearing in the plural encompasses its singular counterpart, unless implicitly or explicitly understood or stated otherwise. Furthermore, it is understood that for any given component or embodiment, any of the possible candidates or alternatives listed for that component may generally be used individually or in combination with one another, unless implicitly or explicitly understood or stated otherwise. Moreover, the figures are not necessarily drawn to scale, wherein some of the elements may be drawn merely for clarity. Also, reference numerals may be repeated among the various figures to show corresponding or analogous elements. Additionally, any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Various embodiments of transgenic biosynthesis of target fatty acids using TAG remodeling are described herein. In the following description, specific details of systems, components, and operations are included to provide a thorough understanding of certain embodiments of the disclosed technology. A person skilled in the relevant art will also understand that the technology may have additional embodiments. The technology may also be practiced without several of the details of the embodiments described below.

As used herein, “TAG remodeling” generally refers to the pathway for oil biosynthesis wherein oil-producing organisms such as seed-oil crops first produce TAG containing common fatty acids, and then change the fatty acid composition of the oil after it is synthesized to accumulate a different fatty acid composition involving new compositions of common fatty acids, or including the valuable unusual fatty acids.

As used herein, fatty acid or TAG “assembly” genes or enzymes generally refer to those associated with building up simpler lipid molecules, such as DAG, into more complex molecules, such as TAG. One example in this category is DGAT1. Assembly genes are distinguished here from those which when expressed contribute to production of lipases, which act to break down complex molecules, such as TAGL1 or a similar TAG lipase.

As used herein, “normal” or “common” fatty acid generally refers to common fatty acids found within oils of most plants, such as the non-HFA portion of castor oil. “Unusual” fatty acids refers to fatty acids produced by select organisms that may have enhanced value for nutrition, bio-fuels, or as chemical feedstocks.

As used herein, the term “gene” refers to an element or combination of elements that are capable of being expressed in a cell, either alone or in combination with other elements. In general, a gene comprises (from the 5′ to the 3′ end): (1) a promoter region, which includes a 5′ non-translated leader sequence capable of functioning in cells; (2) a structural gene or polynucleotide sequence, which codes for the desired protein; and (3) a 3′ non-translated region, which typically causes the termination of transcription and the polyadenylation of the 3′ region of the RNA sequence. Each of these elements is operably linked by sequential attachment to the adjacent element. A gene comprising the above elements is inserted by standard recombinant DNA methods into an expression vector.

### Specific Description

Embodiments of the disclosure provide methods to exploit and enhance TAG remodeling to control the fatty acid composition of oils from plants, protists, bacteria, and fungi for food, bio-fuels, or feedstocks for the chemical industry. Earlier plant oil engineering relied on fatty acid modification and TAG assembly genes from species that accumulate unusual fatty acids. While some of the desired fatty acids have been produced in model species or crop plants, typically the yield of the desired fatty acid is low, and often leads to reduced total oil accumulation as well. Use of lipases to remodel TAG production can be combined with other forms of oil bioengineering utilizing enzymes that produce specific fatty acids and use specific fatty acids for TAG synthesis.

Modification of organisms to enhance lipase expression induces TAG remodeling to change the composition of stored oils. Lipase converts endogenously synthesized TAG back to diacylglycerols, allowing resynthesis into TAG with different fatty acid compositions using fatty acid assembly enzymes either native to the organism or expressed through additional transgenic modification.

Embodiments of the disclosure provide a transgenic method for TAG remodeling of an oil composition of oil-producing plants, protists, bacteria, or fungi to increase production of target fatty acids, comprising modifying one or more cells of a selected oil-producing species by inserting a first foreign gene sequence associated with production of lipase into the one or more cells; cultivating one or more organisms from the one or more modified cells; and selecting a specimen from the one or more organisms, wherein the selected specimen exhibits increased concentration of a target fatty acid as compared to an unmodified control organism of the selected oil-producing species grown under substantially similar growth conditions. Some embodiments further include steps of extracting or collecting the fatty acids produced in the genetically modified species, e.g. via mechanical or solvent extraction.

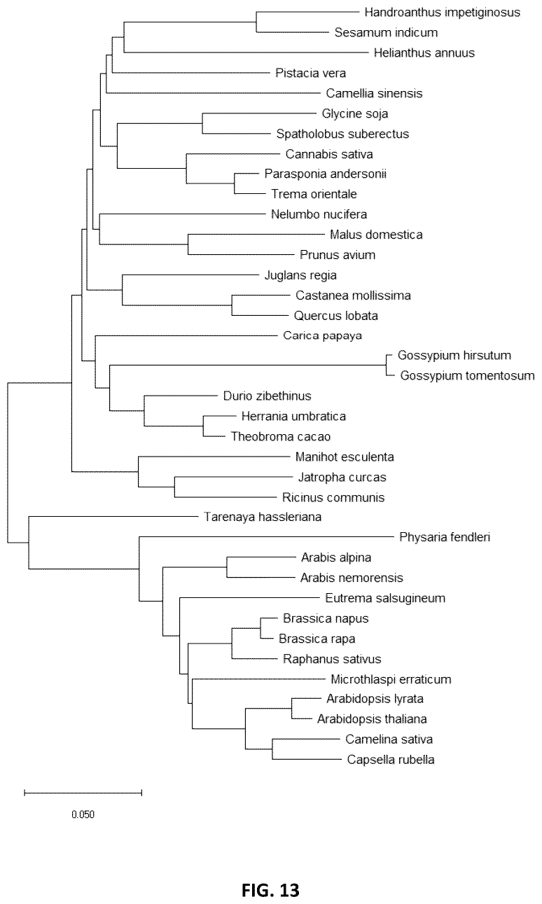

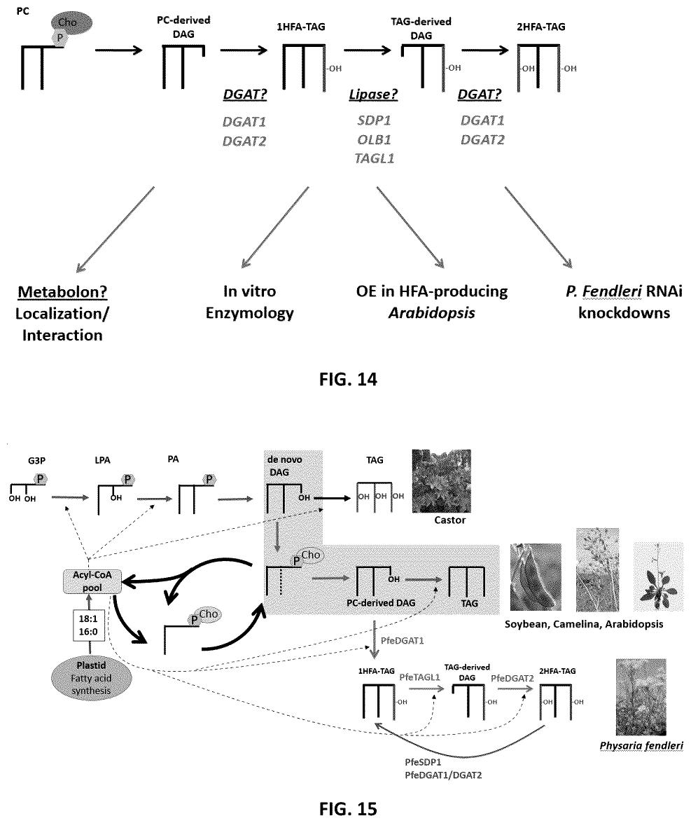

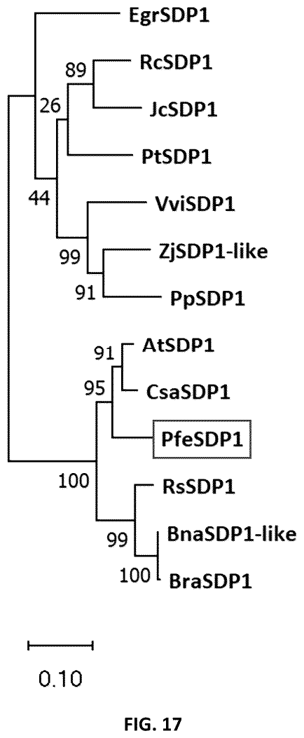

The oil-producing species may include plants, protists, bacteria, or fungi. As used herein, the term “plant” refers to whole plants, plant organs (i.e., leaves, stems, flowers, roots, etc.), seeds and plant cells (including tissue culture cells), and progeny of same. The class of plants that can be used in the method of the disclosure is generally as broad as the class of higher plants amenable to transformation techniques, including both monocotyledonous and dicotyledonous plants, as well as certain lower plants such as algae. Suitable plants include plants of a variety of ploidy levels, including polyploid, diploid and haploid. The term “transgenic plant” refers to a plant modified to express one or more genes. Suitable oil-producing species include, but are not limited to, Physaria fendleri, Ricinus communis, Arabidopsis thaliana, Camelina sativa, Brassica napus, Brassica rapa, Brassica carinata Glycine max, Thlapsi arvense, Linaceae usitatissimum, Helianthus annuus, Sesamum indicum, Cannabis sativa, Crambe abyssinica, Arachis hypogaea, Nicotiana tabacum, Saccharum officinarum, Lemna minor Raphanus sativus, Ziziphus jujuba, Prunus persica, Populus trichocarpa, Jatropha curcas, Vitis vinifera, Eucalyptus grandis, or other species, e.g. as shown in FIGS. 13 and 17. Such species may be genetically engineered to increase expression of a foreign gene or may be the source of the foreign gene. Additional sources of foreign gene include any species that produces unusual fatty acids such as the plant species listed in: Ohlrogge J, Thrower N, Mhaske V, Stymne S, Baxter M, Yang W, Liu J, Shaw K, Shorrosh B, Zhang M, Wilkerson C, Matthaus B (2018) PlantFAdb: a resource for exploring hundreds of plant fatty acid structures synthesized by thousands of plants and their phylogenetic relationships. Plant J 96:1299-1308. It may also include marine organisms, insects, bacteria, fungi, etc. that accumulate unusual fatty acids.

In one embodiment, the genetically modified species has increased expression of a gene encoding a polypeptide selected from the group consisting of a lipase and a TAG assembly enzyme and wherein the gene is operably linked to a promoter. In some embodiments, the lipase is a TAG lipase such as TAG lipase like-1 (TAGL1). An exemplary TAGL1 nucleotide and protein sequence from P. fendleri is provided in SEQ ID NO:1 and SEQ ID NO:2, respectively. In some embodiments, the TAG assembly enzyme is diacylglycerol (DAG) acyltransferase 1 (DGAT1) or diacylglycerol (DAG) acyltransferase 2 (DGAT2). An exemplary nucleotide sequence for DGAT1 and DGAT2 from P. fendleri is provided in SEQ ID NO:3 and SEQ ID NO:4 respectively. An exemplary nucleotide sequence for DGAT1 and DGAT2 from Ricinus communis is provided in SEQ ID NO:5 and SEQ ID NO:6 respectively. In some embodiments, the promoter is selected from the group consisting of constitutive promoters, tissue specific promoter (e.g. developing seeds), regulatable promoters, and inducible promoters. In another embodiment, the promoter is selected from the group consisting of albumin, beta-conglycinin, CaMV 35S, Rubisco, a histone gene promoter, ubiquitin, criptic tCUP, VR-ACSI, CsVMV, ScBV, cLF4A-10, and ibAGP1.

In some embodiments, the foreign gene is substantially homologous to a sequence as provided in SEQ ID Nos 1-6. As disclosed herein, “substantially homologous sequences” include those sequences which have at least about 50%, homology, preferably at least about 60%, more preferably at least about 70% homology, even more preferably at least about 80% homology, and most preferably at least about 90%, 91%, 92%, 93%, 94%, 95%, 96%, 97%, 98%, 99% or more homology to the sequences of the disclosure. In other words, the foreign gene sequences may have at least about 50%, 65%, 70%, 75%, 80%, 85%, 90%, 95%, 96%, 97%, 98%, 99% or more sequence identity to the polynucleotides or a transcriptionally active fragment thereof, or a polypeptide encoded by the polynucleotides. To determine the percent identity of two amino acid sequences or two nucleic acid sequences, the sequences are aligned for optimal comparison purposes (i.e., gaps can be introduced in the sequence of a first amino acid or nucleic acid sequence for optimal alignment with a second nucleic acid sequence). The amino acid residue or nucleotides at corresponding amino acid or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=#of identical overlapping positions/total #of positions×100). In one embodiment, the two sequences are the same length.

Variations in the structure of the polypeptides may arise naturally as allelic variations, as disclosed above, due to genetic polymorphism, for example, or may be produced by human intervention (i.e., by mutagenesis of cloned DNA sequences), such as induced point, deletion, insertion and substitution mutants. Minor changes in amino acid sequence are generally preferred, such as conservative amino acid replacements, small internal deletions or insertions, and additions or deletions at the ends of the molecules.

Substitutions may be designed based on, for example, the model of Dayhoff et al. (1978) Atlas of Protein Sequence and Structure, Natl. Biomed. Res. Found. Washington, D.C. These modifications can result in changes in the amino acid sequence, provide silent mutations, modify a restriction site, or provide other specific mutations.

Specifically designed expression vectors can allow the shuttling of DNA between hosts, such as between bacteria and plant cells. According to one embodiment, the expression vector contains an origin of replication for autonomous replication in host cells, selectable markers, a limited number of useful restriction enzyme sites, active promoter(s), and additional regulatory control sequences.

Preferred among expression vectors, in certain embodiments, are those expression vectors that contain cis-acting control regions effective for expression in a host operatively linked to the polynucleotide of the disclosure to be expressed. Appropriate trans-acting factors are supplied by the host, supplied by a complementing vector or supplied by the vector itself upon introduction into the host. In certain preferred embodiments in this regard, the expression vectors provide for specific expression. Such specific expression is an inducible expression, cell or organ specific expression, host-specific expression, or a combination thereof.

In one embodiment, the expression vector is an Agrobacterium-based expression vector. Various methods are known in the art to accomplish the genetic transformation of plants and plant tissues by the use of Agrobacterium-mediated transformation systems, i.e., A. tumefaciens and A. rhizogenesis.

Gene constructs of the present disclosure can also include other optional regulatory elements that regulate, as well as engender, expression. Generally such regulatory control elements operate by controlling transcription. Examples of such regulatory control elements include, for example, enhancers (either translational or transcriptional enhancers as may be required), repressor binding sites, terminators, leader sequences, and the like.

The integration of the polynucleotides encoding the desired gene into the host is achieved through strategies that involve, for example, insertion or replacement methods. These methods involve strategies utilizing, for example, direct terminal repeats, inverted terminal repeats, double expression cassette knock-in, specific gene knock-in, specific gene knock-out, random chemical mutagenesis, random mutagenesis via transposon, and the like. The expression vector is, for example, flanked with homologous sequences of any non-essential plant genes, bacteria genes, transposon sequence, or ribosomal genes. Preferably the flanking sequences are T-DNA terminal repeat sequences. The DNA is then integrated in host by homologous recombination occurred in the flanking sequences using standard techniques.

Embodiments of the disclosure further provide transgenic species, e.g. plants, including whole plants, plant organs (i.e., leaves, stems, flowers, roots, etc.), seeds and plant cells (including tissue culture cells), and progeny of same that are transformed with a gene construct according to this disclosure. Further embodiments provide transgenic bacteria, fungi, algae, etc.

The selected transgenic specimen exhibits increased concentration of total fatty acids or a target fatty acid as compared to an unmodified control organism of the selected oil-producing species grown under substantially similar growth conditions. For example, the selected specimen has at least 2, 3, 4, 5, 6, 7, 8, 9, or 10-fold the amount by weight of total or target fatty acids. For example, the TAG remodeling process involved in expression of a lipase or a lipase associated with DGAT1 and/or DGAT2 can provide healthy beneficial changes in seed oil content including but not limited to increased omega-3 fatty acids and decreased omega-6 fatty acids and reductions in saturated fatty acids, or when associated with unusual fatty acid synthesis enzymes the enhanced accumulation of unusual fatty acids, and enhanced total oil.

Once plant cells have been transformed, there are a variety of methods for regenerating plants. The particular method of regeneration will depend on the starting plant tissue and the particular plant species to be regenerated. In general, transformed plant cells are cultured in an appropriate medium, which contain selective agents such as, for example, antibiotics, where selectable markers are used to facilitate identification of transformed plant cells. Once callus forms, embryo or shoot formation are encouraged by employing the appropriate plant hormones in accordance with known methods, and the shoots transferred to rooting medium for regeneration of plants. The plants are then used to establish repetitive generations, either from seeds or using vegetative propagation techniques. The presence of a desired gene, or gene product, in the transformed plant may be determined by any suitable method known to those skilled in the art. Included in these methods are southern, northern, and western blot techniques, ELISA, DNA sequencing, PCR, qPCR, and bioassays.

While the present invention has been illustrated by the description of embodiments thereof and specific examples, and while the embodiments have been described in considerable detail, it is not intended to restrict or in any way limit the scope of the appended claims to such detail. Additional advantages and modifications will readily appear to those skilled in the art. Thus, the invention in its broader aspects is therefore not limited to the specific details, representative apparatus and methods and illustrative examples shown and described. Accordingly, departures may be made from such details without departing from the scope or spirit of applicant's general inventive concept.

It is to be understood that this invention is not limited to particular embodiments described herein above and below, and as such may, of course, vary. It is also to be understood that the terminology used herein is for the purpose of describing particular embodiments only, and is not intended to be limiting.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. at which the cell reaction takes place

The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Example

To better understand and characterize enzymes and genes involved in TAG remodeling, P. fendleri was selected for further study because it produces unusual fatty acids (e.g., HFA) valuable for the chemical industry, along with model plant Arabidopsis thaliana, and the oilseed crop species Camelina sativa both of which in unmodified specimens does not accumulate significant HFA. In vivo isotopic tracing of lipid metabolism was used to help characterize the mechanisms of how P. fendleri produce TAG, but by itself cannot indicate which genes perform the reactions.

TAG remodeling in P. fendleri involves removal of non-hydroxylated fatty acid from initially formed TAG species and addition of HFAs to make the final TAG molecule with two HFA. Based on previous transcriptomics data from P. fendleri developing seeds and recent enzymatic assays, protein-protein interactions studies, and subcellular localization studies, P. Fendleri TAG lipase like-1 (TAGL1, PfeTAGL1) and PfeDGAT1-1 and/or PfeDGAT2 were identified as candidates to be the TAG lipase and the DGAT enzyme working together for P. fendleri TAG remodeling. PfeTAGL1 is the only TAG lipase that interacted with PfeDGAT1-1 in both yeast two-hybrid (Y2H) and bimolecular fluorescence complementation (BiFC) assays. To better understand the role of these enzymes in HFA-TAG biosynthesis, P. fendleri DGATs and PfeTAGL1 were expressed in Arabidopsis expressing castor fatty acid hydroxylase (RcFAH) that produces ˜15% HFA in its seed TAG. Transgenic lines overexpressing PfeDGAT1-1, PfeDGAT2 or PfeTAGL1 by itself were made to study their individual role in TAG assembly. In addition to those lines, PfeTAGL1 was also co-expressed with either PfeDGAT1-1 or PfeDGAT2 enzymes individually, and with all three genes together to confirm whether these genes play a role in TAG remodeling and can reconstitute that mechanism in a different plant species.

Representative genes involved in TAG remodeling have now been confirmed within Arabidopsis and Camelina, and include a previously uncharacterized gene for PfeTAGL1. Because lipases are known to break down TAG and reduce seed oil accumulation, involvement of a lipase in increasing accumulation of target fatty acids is counterintuitive. Though known in the field, this effect was confirmed by expressing the P. fendleri homolog of sugar-dependent TAG lipase 1 (SDP1) by itself, which led to less seed oil, as expected. However, the PfeTAGL1 led to both increased seed oil and increased accumulation of valuable hydroxylated fatty acids (HFA) when TAGL1 was expressed with a fatty acid hydroxylase from R. communis in A. thaliana. Thus, the TAGL1 gene has a different role in seed oil metabolism than what is known about lipases such as SDP1.

Further characterization of TAGL1 through enzymatic, protein-protein interaction, and cell biology studies has provided a mechanism of how TAG remodeling functions. Unlike lipases involved in oil breakdown that localize to oil bodies, TAGL1 localized to the endoplasmic reticulum where TAG synthesis from DAG acyltransferase (DGAT) enzymes takes place, and TAGL1 directly interacts with DGAT1 to be able to utilize newly synthesized TAGs. PfeTAGL1 was also demonstrated to have a fatty acid selectivity to remove common fatty acids from TAG rather than the valuable HFA that accumulate in P. fendleri. Thus, PfeTAGL1 can remove fatty acids from newly synthesized TAG, forming DAG which can then be converted back to a different form of TAG, thus changing the oil fatty acid composition.

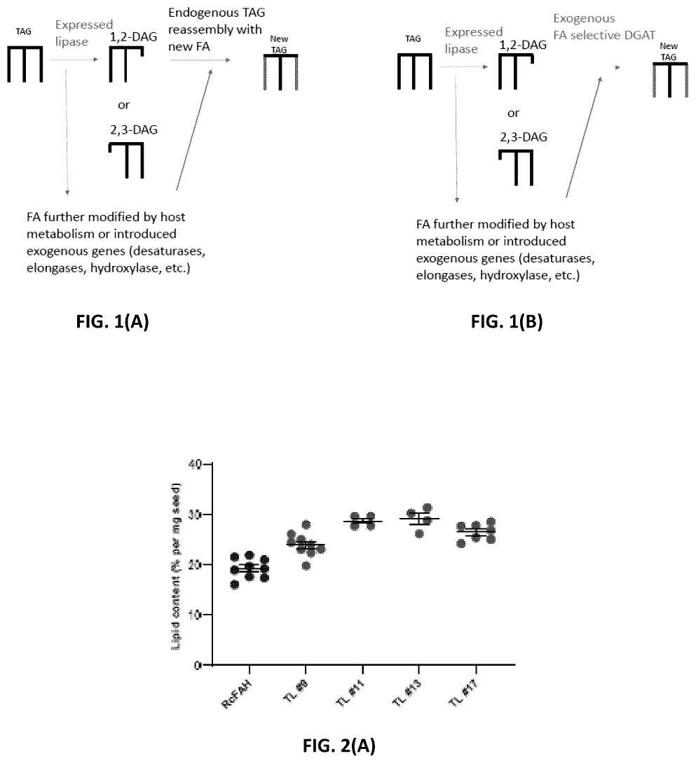

TAG remodeling has now been shown in a representative embodiment to overcome bottlenecks by increasing both the desired fatty concentration and increase total oil accumulation. TAG lipases heterologously expressed during the oil accumulation phase of an oil accumulating plant (or algae, or fungi, or bacteria) have been shown to convert the endogenously synthesized TAG back to DAG allowing resynthesis of TAG with a new fatty acid composition, thus inducing TAG remodeling. The fatty acid released can then reenter lipid metabolism for further modification to desired fatty acid compositions and its reutilization in TAG synthesis (FIG. 1(A)). If the lipase expression is combined with fatty acid assembly enzymes that produce unusual fatty acids (e.g., hydroxy, epoxy, ring structures, unusual double bond locations, alternative chain lengths, polyunsaturated fatty acids, omega-3 fatty acids) then this can lead to higher amounts of the desired unusual fatty acids. By combining the lipase, fatty acid assembly genes, and selective TAG genes (e.g. DGAT or PDAT), then this can further enhance the selectivity of TAG remodeling to produce the desired composition (FIG. 1(B)).

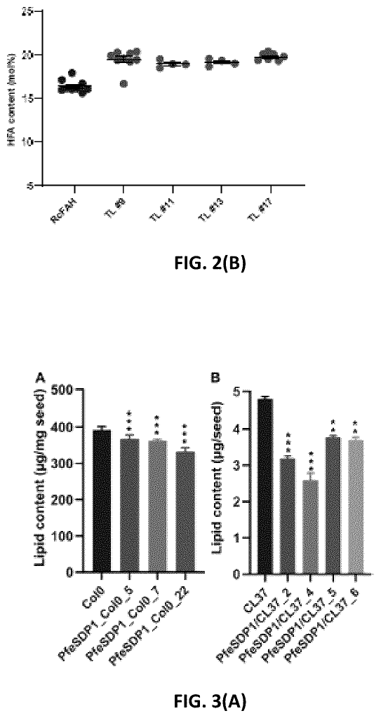

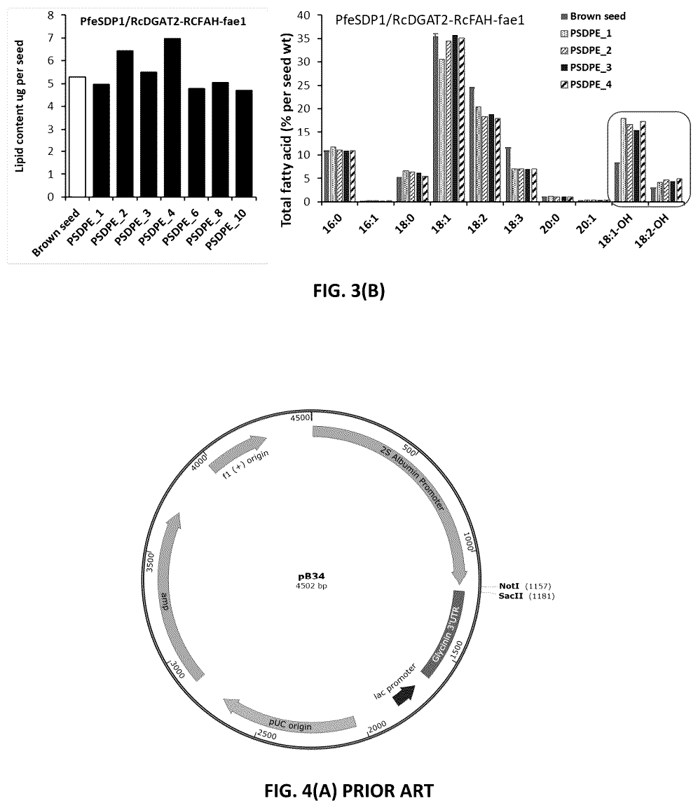

Previously, Arabidopsis seeds engineered to accumulate HFA by expression of the R. communis FAH12 have been demonstrated to accumulate ˜15% HFA but also have a reduced total oil phenotype. FIGS. 2(A) and 2(B) show results demonstrating that the fatty acid selectivity of PfeTAGL1 alone can induce TAG remodeling to increase HFA accumulation and total oil accumulation in the Arabidopsis seeds. However, the lipase does not have to have a fatty acid selectivity against the desired fatty acids. If a general TAG lipase is combined with a fatty acid selective TAG synthesis enzyme, then the DAG produced by the lipase can be used for fatty acid selective TAG synthesis. As shown in FIG. 3(A), the expression of P. fendleri SDP1 alone reduced oil accumulation, however when it was co-expressed with the HFA selective TAG synthesis enzyme R. communis DGAT2, both the amount of oil and levels of HFA increased beyond that of just expressing the DGAT2 alone (FIG. 3(B)).

### Research Approach

After recently recognizing the potential of TAG remodeling, the research team looked at TAG remodeling via tandem action of TAG lipase and DGAT enzyme to replace a portion of normal fatty acid with HFA. Protein-protein interaction analysis and RNAi gene knockdowns in Physaria fendleri suggested PfeDGAT1-1 PfcDGAT2 and PfeTAGL1 might be the enzymes involved in TAG remodeling, prompting continued work on characterizing roles in HFA accumulation.

Transgenic Arabidopsis that accumulates HFA in its seed TAG was used as a background to overexpress P. fendleri genes. Most of the transgenic lines with P. fendleri genes increased amounts of total lipid as well as HFA.

Lipid biosynthetic genes from plants such as R. communis and P. fendleri which produce HFA in their seed oil TAG have been unrealized targets for bioengineering other oilseeds such as Arabidopsis thaliana and Camelina sativa. However, previous transgenic lines utilizing multigene engineering strategies have only achieved accumulated levels of HFA <40% in Arabidopsis and <25% in Camelina which compares unfavorably to plants like R. communis (˜90%) and P. fendleri (˜60%) which produce HFAs without modification. Thus, one of the major constraints in engineering these unusual fatty acids has been a lack of full understanding about the TAG assembly pathway and genes required for accumulation of HFAs in seed TAG. Therefore, characterizing the genes involved in TAG remodeling is important in improving the production of HFAs in Camelina as well as enhancing P. fendleri as a crop plant.

### Research Results

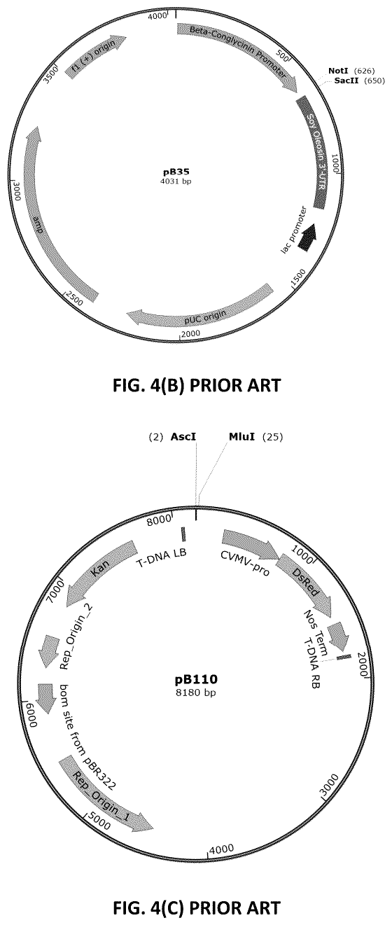

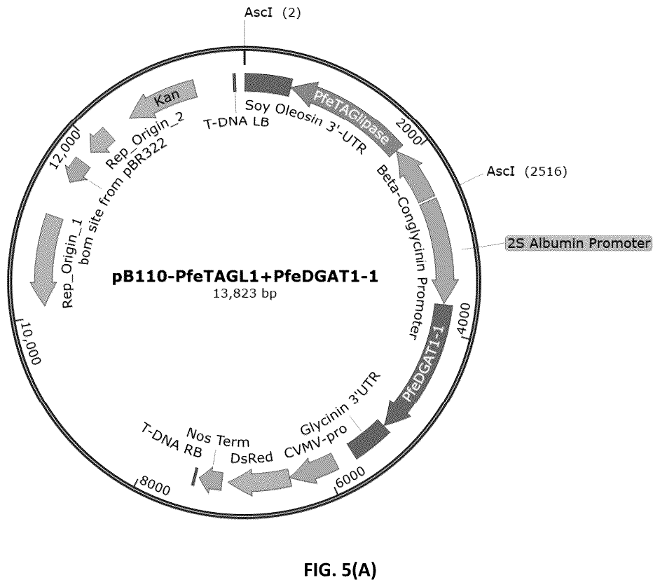

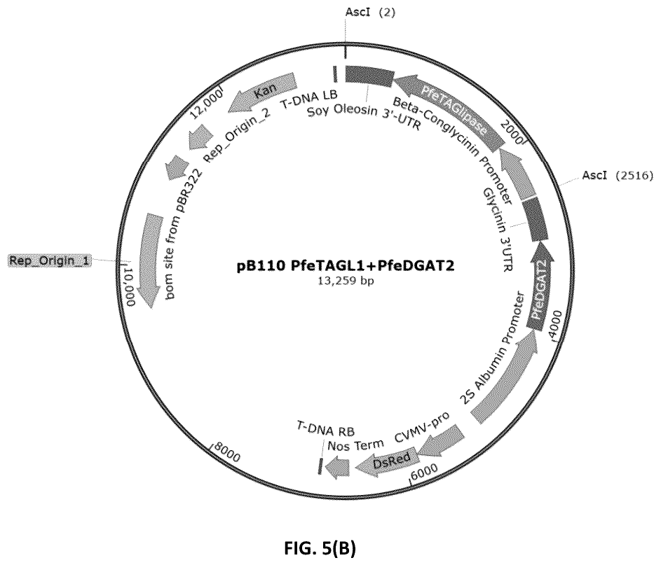

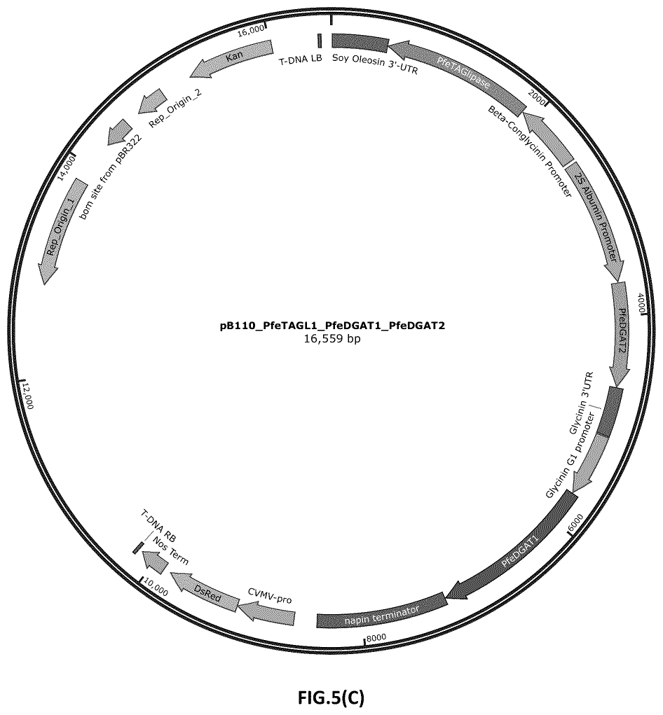

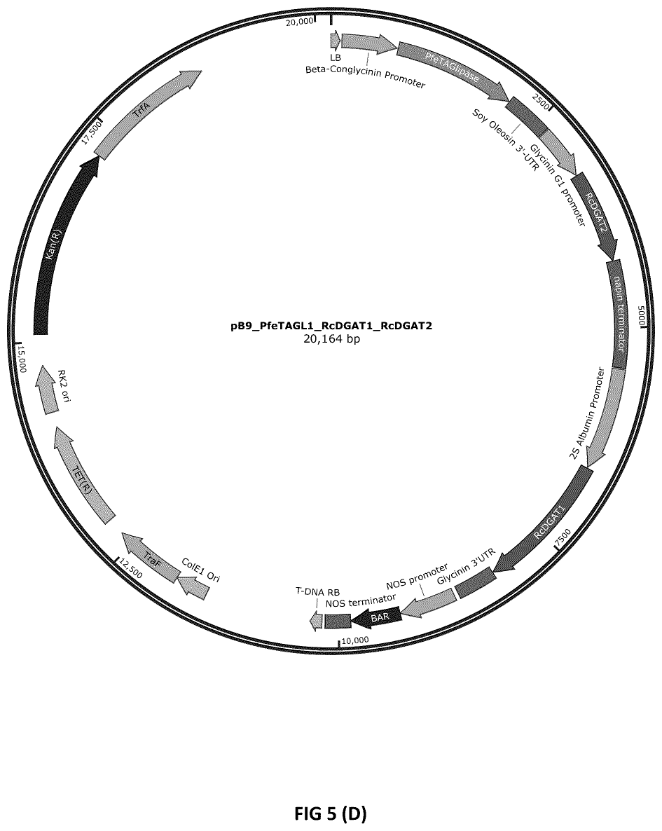

P. fendleri genes introduced in A. thaliana HFA line increased total lipid and HFA content. All the genes/gene combinations were expressed under seed specific promoter in this study to determine the role of P. fendleri genes in HFA TAG assembly pathway in seeds. Both the DGATs, PfeDGAT1-1 or PfeDGAT2 were expressed under the seed specific At2S-albumin promoter and glycinin terminator in pB34 vector (FIG. 4(A)) and PfeTAGL1 was expressed under the seed specific beta-conglycinin promoter and soy oleosin terminator in pB35 vector (FIG. 4(B)). These gene cassettes were put into pB110 binary vector with dsRed fluorescence marker which allows to select positive transformant seeds that fluoresces red under green light. For two gene stacked lines, gene cassette of PfeTAGL1 was inserted in head-to-head orientation with PfeDGAT1-1 (FIG. 5(A)) or PfeDGAT2 (FIG. 5(B)). For three gene stacks the PfeTAGlipasc used the beta-conglycinin promoter, the PfcDGAT2 had the 2s Albumin promoter and the PfcDGAT1 had the Glycinin GI promoter (FIG. 5(C)).

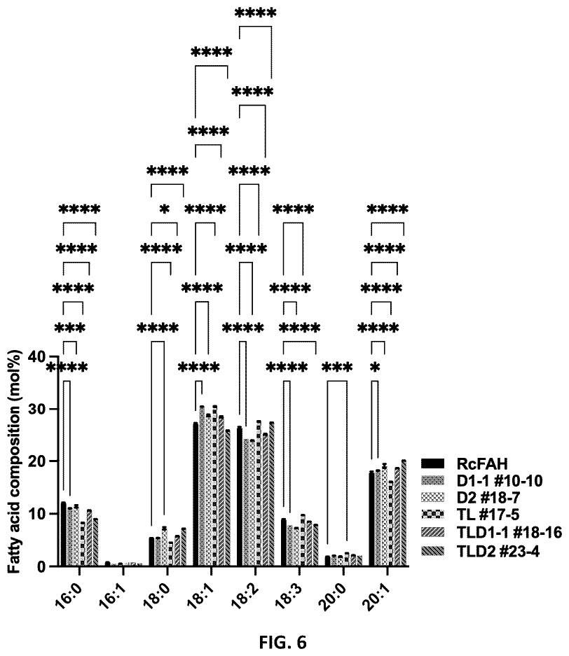

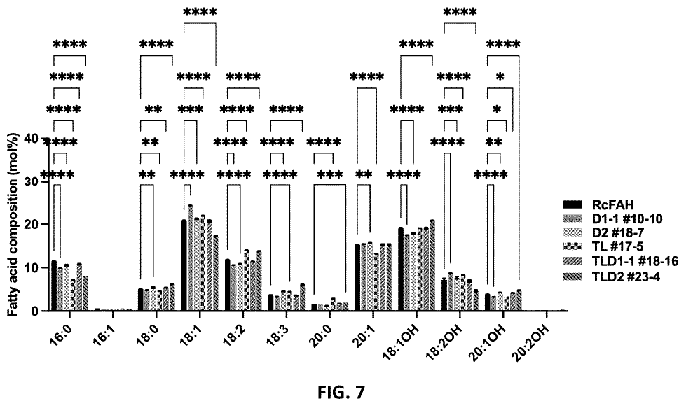

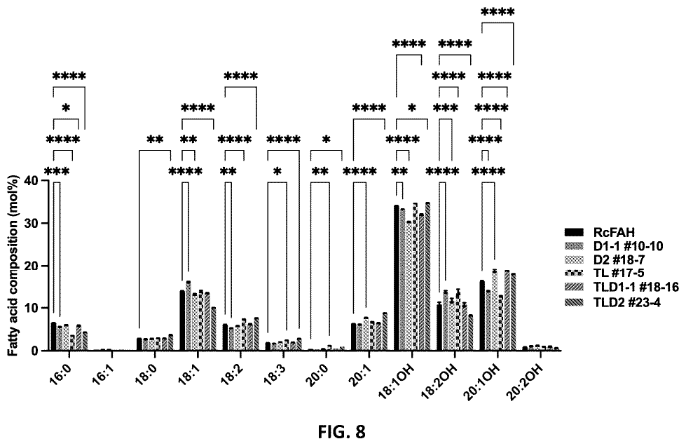

Groups of TAG molecular species corresponding to 0-, 1-, or 2-HFA were separated and the fatty acid content analyzed. In PfeDGAT1-1 line, 16:0, 18:2, 18:3 decreased whereas 18:1 and 20:1 increased in the regular TAG fraction without HFA (FIG. 6). Effect of PfeDGAT2 in regular TAG is similar to PfeDGAT1-1, the only difference is the increase in 18:0 fatty acid (FIG. 6). In case of line overexpressing PfeTAGL1, amount of both 16 C and 18 C saturates and 20:1 decreased but there is an increase in 18 C monounsaturated and polyunsaturated fatty acids (FIG. 6) indicating PfeTAGL1 might selectively cleave off saturates and 20:1 fatty acid to allow more HFA (or polyunsaturated fatty acids) to be incorporated. Double stack lines TLD1-1 and TLD2 show similar changes in TAG composition, the only difference being TLD1-1 increased 18:1 but TLD2 increased 18:2 instead (FIG. 6). Most of the effects of P. fendleri genes in 1HFA-TAG and 2HFA-TAG are similar to regular TAG species when non-hydroxy fatty acids were analyzed (FIG. 6-8). For HFA composition in 1HFA-TAG species, 18:1OH increased in the TLD2 line whereas 18:2OH increased in the D1-1 and TL lines (FIG. 7). Lesquerolic acid (20:1OH) increased in PfeDGAT2 lines either by itself (D2) or with PfeTAGL1 (TLD2) compared to RcFAH (FIG. 7). However, in the case of 2HFA-TAG, there is a significant increase in lesquerolic acid (20:1OH) in PfeDGAT2 by itself as well as both stacked lines TLD1-1 and TLD2 (FIG. 8). Increase in 20:1 as well as 20:10H in PfeDGAT2 and PfeTAGL1+PfeDGAT2 in different TAG species indicates that PfeDGAT2 can incorporate 20C fatty acids, both HFA and non-HFA (FIG. 6-8). However, PfeDGAT1-1 requires action of PfeTAGL1 to add in 20C HFA as increase in lesquerolic acid (20:1OH) is observed in TLD1-1 line but not in line expressing PfeDGAT1-1 alone (FIG. 8).

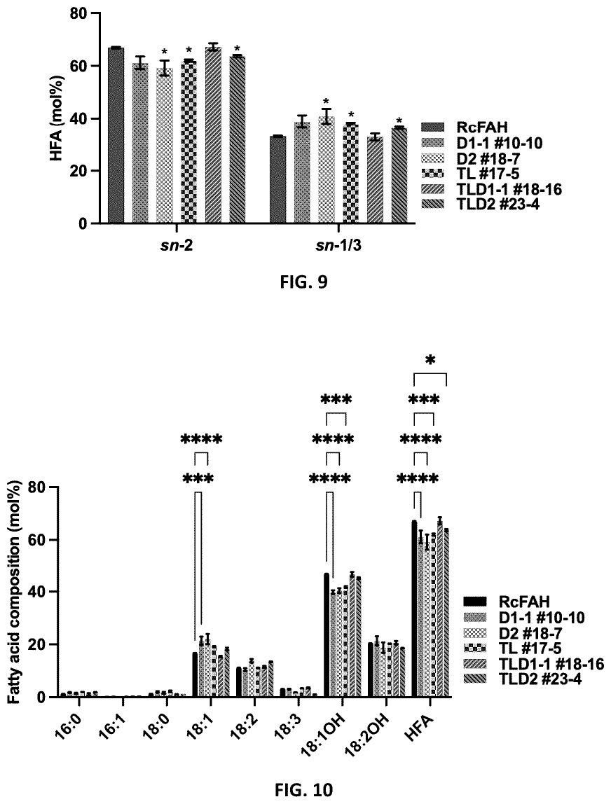

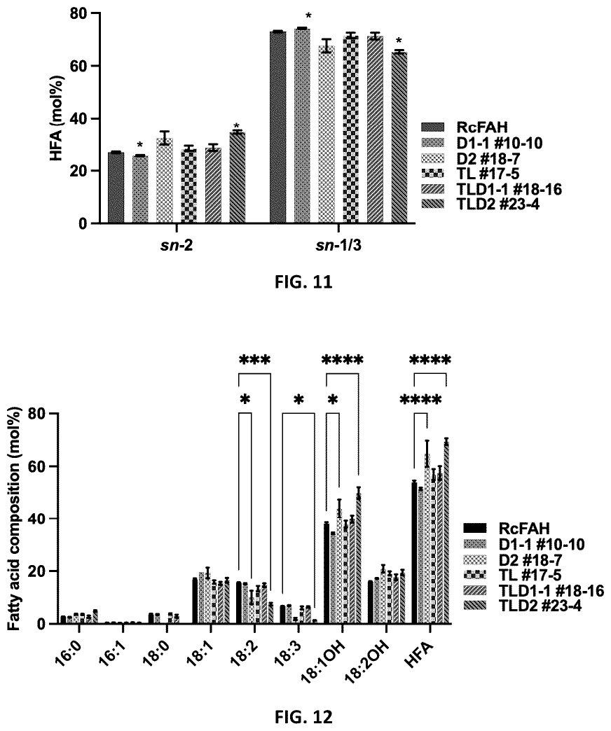

PfeDGAT2 increases HFA incorporation at sn-1/3 position of TAG molecular species containing one HFA. Regiochemical analyses of IHFA-TAG and 2HFA-TAG for all the transgenic lines along with control RcFAH were performed to see any change in the position of HFA in those HFA-TAG molecular species. HFA percentage at sn-2 position was compared to sn-1/3 position in both 1HFA-TAG and 2HFA-TAG. In 1HFA-TAG molecular species, HFA at sn-1/3 was significantly increased in PfeDGAT2, PfeTAGL1 and PfeTAGL1+PfeDGAT2 lines (FIG. 9). However, there were significant decreases in HFA content in MAG from 1HFA-TAG digest of PfeDGAT1-1 as well (FIG. 10). Since fatty acid composition of MAG represents the fatty acid composition at sn-2 position of TAGs, the decrease in HFA in MAGs suggests that HFA content at sn-1/3 position is increased in this line as well (FIG. 10) and p-value for sn-2 HFA of PfeDGAT1-1 was about 0.07 for FIG. 9. Similarly, regiochemical analysis of 2HFA-TAG showed decrease in HFA content at sn-2 position in PfeDGAT1-1 lines whereas increased HFA content at sn-2 position in TLD2 line (FIG. 11). This indicates that HFA at sn-1/3 of 2HFA-TAG is increased in PfeDGAT1-1 line but decreased in PfeTAGL1+PfeDGAT2 lines. It is interesting that 1HFA-TAG (FIG. 7) and 2HFA-TAG (FIG. 8) from all transgenic lines have some amount of 20C HFA, but these 20C HFAs are not present in MAG produced from the digest of those HFA-TAG molecular species (FIGS. 10 and 12). This indicates 20C HFAs are not present at sn-2 position of those HFA-TAGs molecular species. Therefore, increase in lesquerolic acid (20:10H) observed in 2HFA-TAG of PfeDGAT2, PfeTAGL1+PfeDGAT1-1 and PfeTAGL1+PfeDGAT2 lines must be at sn-1/3 position (FIG. 8).

Regiochemical analysis showed that there is an increase in HFA at sn-1/3 (FIG. 9). P. fendleri accumulates HFA-TAGs without HFA at sn-2 position whereas RcFAH line has more than 60% HFA in sn-2 position (FIG. 9) and these DAG species might not be efficiently used by Physaria genes to make HFA-TAGs. Crambe abyssinica DGAT1 utilized 22:1-CoA at very low rate when DAG with 22:1 fatty acid at both sn-1 and sn-2 position was used compared to sn-1-22:1-sn-2-18:1 DAG (DAG available in Crambe seeds). Therefore, HFA present at sn-2 position in Arabidopsis RcFAH line might be limiting the activity of P. fendleri DGATs, and thus combining PfeTAGL1 induced TAG remodeling with DGATs selective for unusual fatty acids at the sn-2 position may further increase unusual fatty acid accumulation.

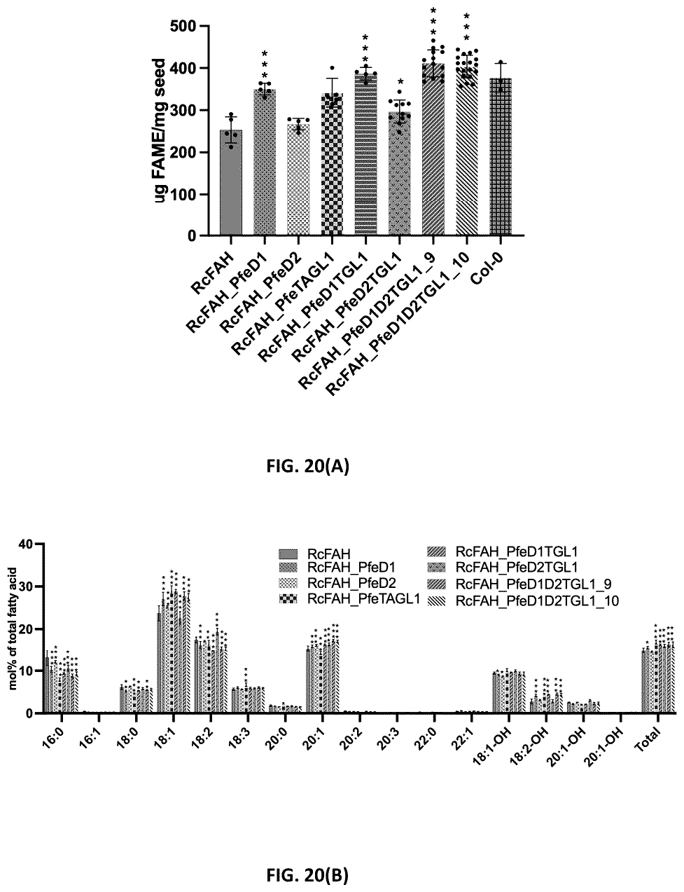

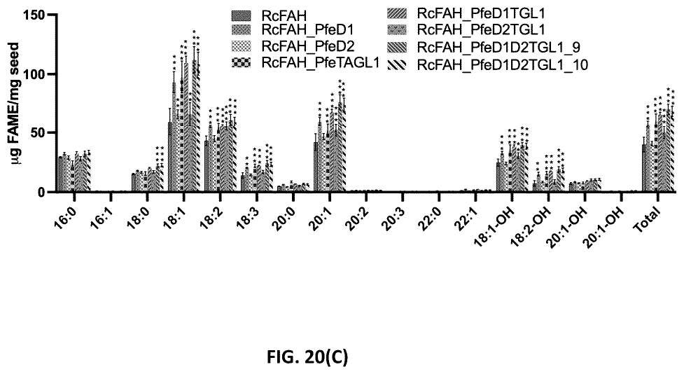

We further demonstrated the effect of combining the three individual TAG remodeling genes from Physaria engineered into Arabidopsis producing HFA (FIGS. 20(A)-(C)). The three genes together eliminate the reduced oil phenotype and have the highest increase in the target fatty acids, demonstrating the additive effect of combining the three TAG remodeling genes together.

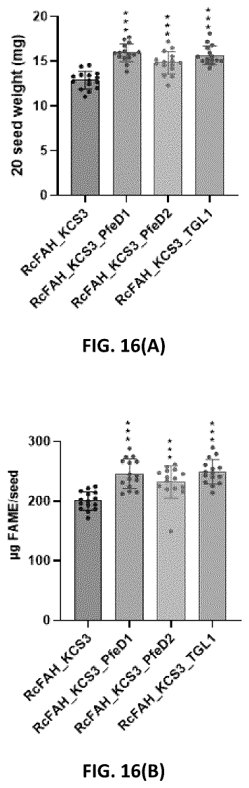

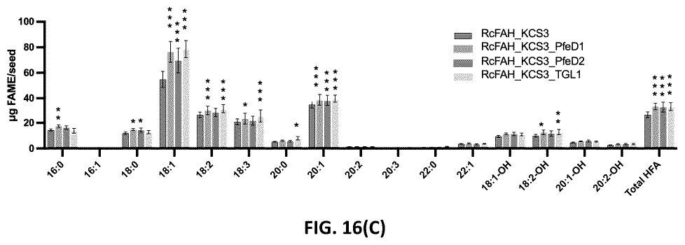

FIGS. 16(A)-(C), 18(A)-19(B) reflect subsequent work confirming effective single TAG remodeling genes in the crop Camelina sativa engineered to produce HFA.

Single TAG remodeling genes can increase seed weight (FIG. 16(A)) and oil content per seed (FIG. 16(B)). in HFA producing Camelina.

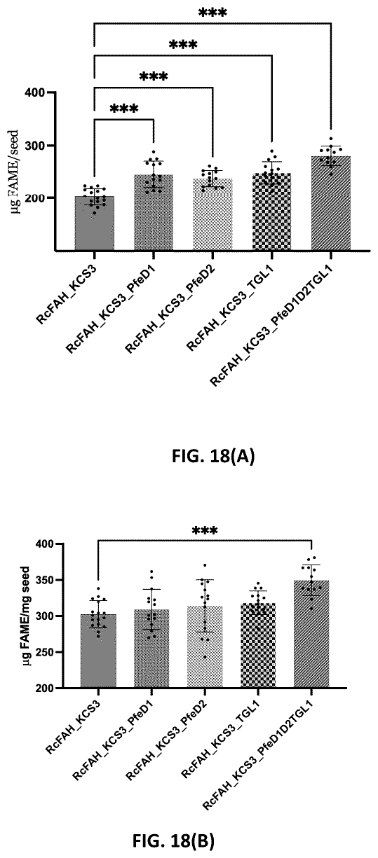

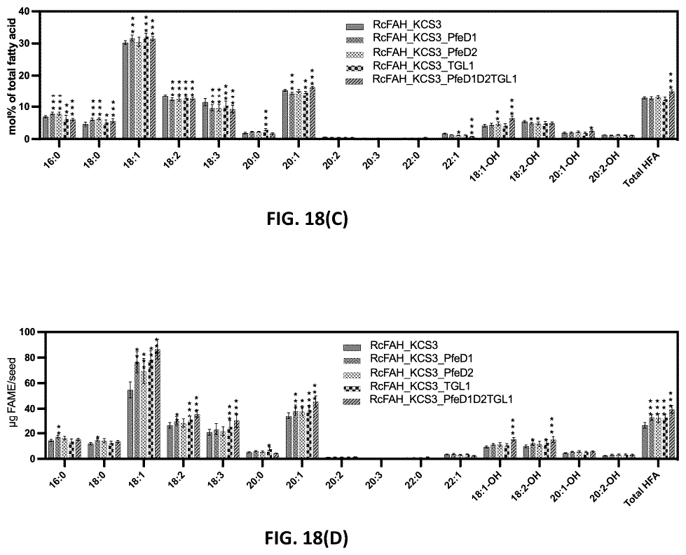

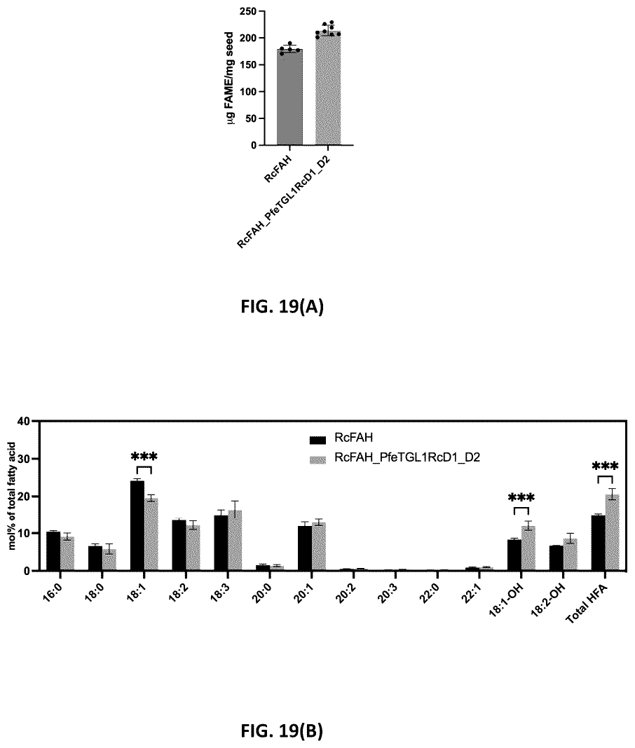

We also demonstrated the effect of the three individual TAG remodeling genes from Physaria engineered into the oilseed crop Camelina sativa line (RcFAH_KCS3) previously engineered to produce 18 carbon and 20 carbon HFA by the castor fatty acid hydroxylase and to produce more 20 carbon lesquerolic acid by 18 carbon ricinoleic acid elongation from the PfeKCS3 (line RcFAH PfeKCS3). Each Physaria gene alone increases oil and HFA content, but by combining all three together the increase in oil and HFA content is the greatest (FIGS. 18(A)-(D)). Thus demonstrating engineering TAG remodeling can work in a crop plant. Additionally, we combined the TAGL1 from Physaria, with the DGAT1 and DGAT2 from Ricinus communis, in a Camelina background that only contained the castor hydroxylase and thus produced mostly 18C ricinolic acid (line RcFAH). Here we demonstrate that we can utilize the TAG remodeling lipase (TAGL1) with TAG synthesizing acyl transferases from a different HFA accumulating species (FIGS. 19(A)-(B)) to further control the type of unusual fatty acid that accumulates.

We then performed triacylglycerol remodeling to change seed oil fatty acid compositions for more nutritious fatty acids including, reduced saturated fatty acids, reduced omega-6 fatty acids, and increased omega-3 fatty acids.

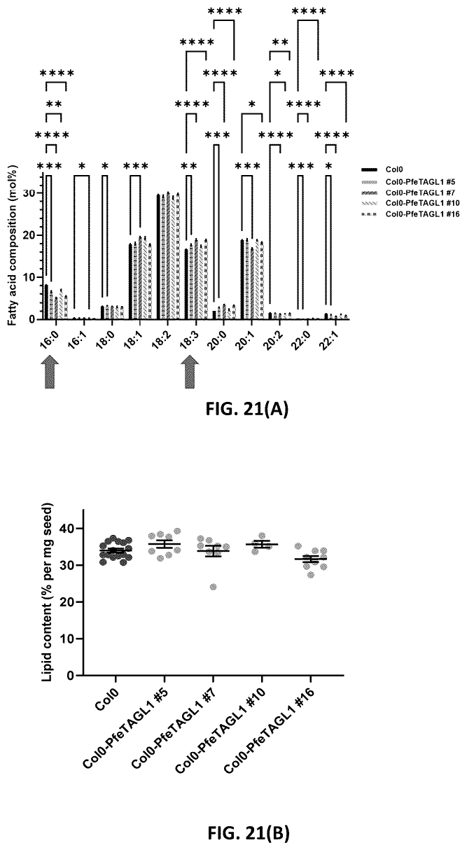

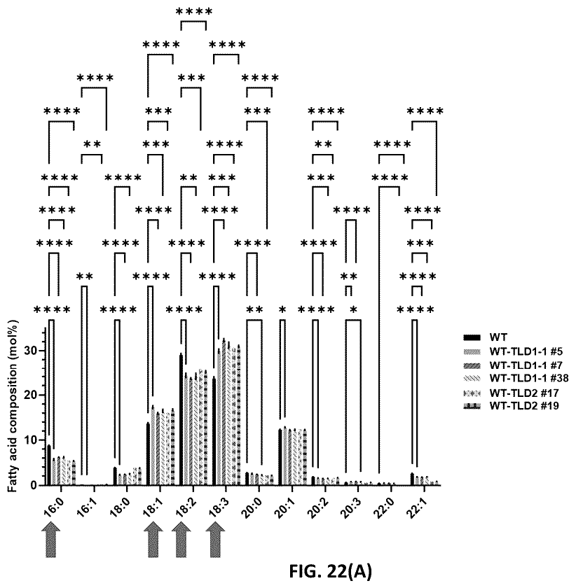

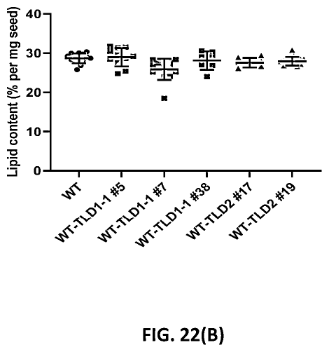

The results demonstrate that the TAG remodeling enzymes from Physaria fendleri can be used to produce desirable fatty acid compositions, even apart from the hydroxylated fatty acids P. fenderli naturally produces. The beneficial changes in seed oil content through engineering TAG remodeling, are demonstrated in Arabidopsis thaliana by the use of the PfeTAGL1 lipase alone (FIGS. 21(A)-(B)), and in Camelina sativa by combining the lipase (TL) with diacylglycerol acyltransferase 1 or 2 (D1 and D2) from P. fendleri (FIGS. 22(A)-(B)). In both species there is a beneficial increase in the omega-3 (18:3)/omega-6 (18:2) ratio and a decrease in saturated fatty acids (16:0). In both plants, the beneficial changes in seed oil fatty acid composition by expression of the lipase do not lead to detrimental reductions in seed oil content.

### Research Methods

Plant growth conditions and supplies. All A. thaliana plant lines were grown in growth chamber set at 22° C. with a light intensity of ˜150-200 μmol m-2 s-1 and 16-hour day and 8-hour night photoperiod. Arabidopsis and Camelina seeds were first surface sterilized with 70% ethanol, 10% bleach with 0.1% sodium dodecyl sulphate (SDS), sterile water and plated on Murashige and Skoog media supplemented with 1% sucrose. After that, plates were vernalized for 3 days at 4° C. and transferred under light for 2 weeks. They were then transplanted to soil and grown until maturity in the growth chamber set at conditions mentioned above. Plants were watered twice a week and provided with fertilizer solution NPK 20-20-20 (0.9957 gL-1) once a week. All the solvents were HPLC grade or above and chemicals were from Fisher Scientific unless indicated.

Cloning and creating transgenic plants. All genes PfeDGAT1-1, PfeDGAT2, PfeTAGL1 were commercially synthesized by Integrated DNA Technology Inc (IDT). PCR using Phusion polymerase was done to create restriction sites 5′NotI and 3′SacII, which were digested with respective enzymes before ligation. All enzymes were from New England Biolabs unless indicated and all plasmids were constructed using plant expression vectors form. To make shuttle plasmids, ORF of PfeDGAT1-1 and PfeDGAT2 were cloned into pB34 with 2S albumin promoter and glycinin terminator (FIG. 4(A)) whereas PfeTAGL1 was cloned into pB35 with beta-conglycinin promoter and soy oleosin terminator (FIG. 4(B)). The promoter: gene: terminator cassettes for these genes were then put into binary vector pB110 (FIG. 4(C)). In addition to plasmids containing PfeDGATs and PfeTAGL1 by itself, binary pB110 vector with PfeTAGL1 and either PfeDGAT1-1 (FIG. 5(A)) or PfeDGAT2 (FIG. 5(B)) or both PfeDGAT1 and PfeDGAT2 (FIG. 5(C)) were constructed in head-to-head and head-tail orientation. Binary pB9 vector with PfeTAGL1 and both RcDGAT1 and RcDGAT2 were constructed in head-to-head and head-tail orientation (FIG. 5(D)). The integrity of all the plasmids were confirmed by sequencing as well as digestion of the plasmid with restriction enzymes before using for agrobacterium and plant transformation. Agrobacterium tumefaciens strain GV3101 was transformed with these different binary vectors method using the freeze-thaw and selected on Rifampicin/Gentamycin/Kanamycin plates and the presence of genes were confirmed by colony PCR. A. thaliana line (RcFAH) and Camelina lines (RcFAH and RcFAH_PfcKCS3) were used as the parental lines which expresses castor fatty acid hydroxylase gene and produced HFA for transformation with agrobacterium containing different binary vectors using floral dip method. Transformed plants were grown to maturity, seeds were harvested and successful transformants were selected based on DsRed fluorescence when observed under green light with red filter on, or based on resistance to Basta herbicide and red T1 seeds or basta resistant seedlings were moved to next generation. Oil content and fatty acid composition of T2 generation was analyzed as mentioned in the section below and top four-six lines were moved to next generation to analyze for homozygosity.

Oil content and fatty acid composition analysis. For total seed oil content and fatty acid composition analysis of T2 and T3 generation of all transgenic lines along with control line RcFAH, about 2-3 mg seeds of Arabidopsis or 20 seeds of Camelina seeds were weighed, 25 μg of 17:0 TAG as internal standard was added, converted into FAMEs using 5% H2SO4 in methanol and heated at 80-85° C. for 1.5 hr. Those FAMEs were extracted with 1 ml hexanes and 1.5 ml 0.88% potassium chloride solution. After centrifugation at 2500 rpm for 2 min, hexanes layer was transferred to GC vial and ran through GC-FID.

Lipid extraction, separation of different lipid species and quantification. Selected T3 homozygous lines were moved to T4 generation, grown until maturity and used for further analysis. About 100 mg seeds from T4 lines were quenched in isopropanol with 0.01% w/v butylated hydroxytoluene (BHT) at 85° C. for 10 min and total lipid was extracted using hexanesisopropanol method and resuspended in toluene. An aliquot of total lipid extracted were converted to fatty acid methyl esters (FAMEs) and quantified by gas chromatography flame ionization detector (GC-FID). Separation of different TAG species were done using HPLC. About 1 mg total lipid resuspended in hexanes: isopropanol (60:40, v/v) was loaded on to YMC-Pack PVA-Sil column at 35° C. Parameter for HPLC gradient method and fraction collection are shown in Table 4.1 and 4.2 respectively. Fractions were dried down under nitrogen, added 25 μg 17:0 TAG as internal standard, converted to FAMEs using 2.5% H2SO4 in methanol at 85° C. for 1 hour, extracted hexane layers were ran through GC-FID for quantification.

Table 1, below, shows HPLC method gradient parameters for separation of different TAG species. Solvents used were A. Isopropanol B. Hexanes C. Methanol and D. Isopropanol: Water: Acetic acid (60/40/0.065, v/v/v).

TABLE 1

| | | |
|---|---|---|
| Time | Flow Rate | Solvent |

| (min) | (ml/min) | % A | % B | % C | % D |
|---|---|---|---|---|---|
| 0.00 | 1.5 | 0.50 | 99.50 | 0.00 | 0.00 |
| 1.00 | 1.5 | 1.00 | 99.00 | 0.00 | 0.00 |
| 4.00 | 1.5 | 1.00 | 99.00 | 0.00 | 0.00 |
| 8.00 | 1.5 | 2.50 | 97.00 | 0.50 | 0.00 |
| 10.00 | 1.5 | 2.50 | 97.00 | 0.50 | 0.00 |
| 12.00 | 1.5 | 5.00 | 94.00 | 1.00 | 0.00 |
| 16.00 | 1.5 | 7.00 | 92.00 | 1.00 | 0.00 |
| 21.00 | 1.5 | 95.00 | 5.00 | 0.00 | 0.00 |
| 22.00 | 1.5 | 50.00 | 0.00 | 25.00 | 25.00 |
| 25.00 | 1.5 | 50.00 | 0.00 | 25.00 | 25.00 |
| 26.00 | 1.5 | 100.00 | 0.00 | 0.00 | 0.00 |
| 29.00 | 1.5 | 100.00 | 0.00 | 0.00 | 0.00 |
| 30.00 | 1.5 | 0.50 | 99.50 | 0.00 | 0.00 |
| 35.00 | 1.5 | 0.50 | 99.50 | 0.00 | 0.00 |

Table 2, below, shows HPLC fraction collection parameters for different TAG molecular species.

TABLE 2

| | |
|---|---|
| Fraction | Fraction collection time |

| No. | Start (min) | End (min) | Lipid species |
|---|---|---|---|
| 1 | 1.92 | 3.40 | TAG |
| 2 | 5.15 | 6.65 | 1HFA-TAG |
| 3 | 11.35 | 12.85 | 2HFA-TAG |

Regiochemical analysis of HFA containing TAG species. For regiochemical analysis, about 9 mg of total lipid was used for bulk collection of 1HFA-TAG and 2HFA-TAG species using HPLC as described above. 1HFA-TAG and 2HFA-TAG fractions were dried under nitrogen and resuspended in toluene. An aliquot of it was used to make FAMEs and run through GC for quantification. 1 mg of each HFA-TAGs were resuspended in 1 ml diethyl ether, 800 μl 50 mM sodium borate buffer (pH 7.6) with 5 mM calcium chloride and 200 μl Rhizomucor meihei lipase (Sigma) were added. TAG digestion was done by shaking those tube vigorously for 45 min at room temperature and 2 ml methanol: chloroform (1:1, v/v) was added to stop the reaction. Lower chloroform layer was collected, dried down and resuspended in 100 μl chloroform. These digested samples were then loaded on to TLC plate and separated using chloroform:methanol: acetic acid (98:2:0.5, v/v/v) solvent system, stained with 0.005% primulin in acetone: water (80:20, v/v) and visualized under UV light. MAG and TAG bands were scrapped off from TLC plates, added 25 μg 17:0 TAG as internal standard and made FAMEs using 2.5% H2SO4 in methanol, heating at 85° C. for an hour. Hexanes layers were extracted and quantified by GC-FID as described above. The mol percentage of HFAs in MAG represents mol % HFA at sn-2 position of TAGs. For determination of HFA % in the sn-1 and sn-3 positions, following formula was used: for 1HFA-TAG, % HFA at the sn-1 and sn-3 positions=(100-% HFA at sn-2) and for 2HFA-TAG, %

HFA at the sn-1 and sn-3 positions=[100−(% HFA at sn-2/2)].

Table 3, below, shows PfeSDP1 homolog species abbreviations and accession numbers for each protein as listed in FIG. 17.

TABLE 3

| Accession | Species | Abbreviation |
|---|---|---|
| This study | Physaria fendleri | PfeSDP1 |
| NP_001332745.1 | Arabidopsis thaliana | AtSDP1 |
| XP_010452294.1 | Camelina sativa | CsaSDP1 |
| XP_013668385.1 | Brassica napus | BnaSDP1 |
| XP_009122642.1 | Brassica rapa | BraSDP1 |
| XP_018446336.1 | Raphanus sativus | RsSDP1 |
| EEF32280.1 | Ricinus communis | RcSDP1 |
| XP_015898084.1 | Ziziphus jujuba | ZjSDP1 |
| XP_007203312.1 | Prunus persica | PpSDP1 |
| XP_002308909.1 | Populus trichocarpa | PtSDP1 |
| XP_012085968.1 | Jatropha curcas | JcSDP1 |
| RVW92138.1 | Vitis vinifera | VviSDP1 |
| XP_039163397.1 | Eucalyptus grandis | EgrSDP1 |

Although the description here contains many details, these should not be construed as limiting the scope of the disclosure but as merely providing illustrations of some of the presently preferred embodiments. Therefore the scope of the disclosure encompasses other embodiments which may become obvious to those skilled in the art.

In these claims, reference to an element in the singular is not intended to mean “one and only one” unless explicitly stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element is to be construed as a “means plus function” element unless the element is expressly recited using the phrase “means for . . . ” No claim element is to be construed as a “step plus function” element unless the element is expressly recited using the phrase “step for . . . .”

It should be emphasized that the above-described embodiments and specific examples of the present invention, particularly, any “preferred” embodiments, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

What is claimed:

1. A transgenic method for triacylglycerol (TAG) remodeling of an oil composition of an oil-producing species to increase production of target fatty acids, comprising:
modifying one or more cells of a selected oil-producing species by inserting a first foreign gene sequence associated with production of lipase into the one or more cells;
cultivating one or more organisms from the one or more modified cells; and
selecting a specimen from the one or more organisms, wherein the selected specimen exhibits increased concentration of a target fatty acid as compared to an unmodified control organism of the selected oil-producing species grown under substantially similar growth conditions.

2. The method of claim 1, wherein the first foreign gene sequence is TAG lipase like-1 (TAGL1).

3. The method of claim 1, wherein the first foreign gene sequence is obtained from Physaria fendleri or Ricinus communis.

4. The method of claim 1, wherein the target fatty acid is one or more hydroxy fatty acid(s) (HFA).

5. The method of claim 1, wherein the selected oil-producing species is Arabidopsis thaliana or Camelina sativa.

6. The method of claim 1, further comprising:
modifying the one or more cells of the selected oil-producing species by inserting a second foreign gene sequence associated with production of at least one TAG assembly enzyme into the one or more cells.

7. The method of claim 6, wherein the second foreign gene sequence is one or more of diacylglycerol (DAG) acyltransferase 1 (DGAT1) and DAG acyltransferase 2 (DGAT2).

8. The method of claim 7, wherein the second foreign gene sequence is obtained from Physaria fendleri or Ricinus communis.

9. The method of claim 1, wherein the selected specimen exhibits one or more of increased omega-3 fatty acids, decreased omega-6 fatty acids, and reduced saturated fatty acids as compared to the unmodified control organism.

10. A transgenic oil-producing species, comprising:
an oil-producing organism modified with a first foreign gene sequence associated with production of lipase, wherein overexpression of the first foreign gene sequence results in increased concentration of at least one target fatty acid as compared to an unmodified control organism of the same species grown under substantially similar growth conditions.

11. The transgenic oil-producing species of claim 10, wherein the oil-producing organism is Arabidopsis thaliana or Camelina sativa.

12. The transgenic oil-producing species of claim 10, wherein the first foreign gene sequence is TAGL1.

13. The transgenic oil-producing species of claim 10, wherein the first foreign gene sequence is obtained from Physaria fendleri or Ricinus communis.

14. The transgenic oil-producing species of claim 10, wherein the target fatty acid is one or more HFA.

15. The transgenic oil-producing species of claim 10, further modified with a second foreign gene sequence associated with production of at least one TAG assembly enzyme.

16. The transgenic oil-producing species of claim 15, wherein the second foreign gene sequence is one or more of DGAT1 and DGAT2.

17. The transgenic oil-producing species of claim 16, wherein the second foreign gene sequence is obtained from Physaria fendleri or Ricinus communis.

18. The transgenic oil-producing species of claim 10, wherein the oil-producing organism exhibits one or more of increased omega-3 fatty acids, decreased omega-6 fatty acids, and reduced saturated fatty acids as compared to the unmodified control organism.
